(19) United States
(12) Patent Application Publication
(10) Pub. No.: US 2024/0159731 A1
(43) Pub. Date: May 16, 2024

(54) Accurate Monitoring of Concrete with a Cast-In-Place Device

(71) Applicants: Malcolm Lim, BUFFALO GROVE, IL (US); Andrew Haapapuro, Arlington Heights, IL (US)

(72) Inventors: Malcolm Lim, BUFFALO GROVE, IL (US); Andrew Haapapuro, Arlington Heights, IL (US)

(21) Appl. No.: 17/816,409

(22) Filed: Jul. 30, 2022

(51) Int. Cl. G01N 33/38

(52) U.S. Cl. CPC G01N 33/383

(57) ABSTRACT

Measuring the relative humidity of concrete is a complex process. ASTM F2170 is currently the standard for embedded monitoring relative humidity in newly placed concrete. The process requires a hole to be drilled and cleaned before inserting a relative humidity probe to begin monitoring relative humidity. The measurements are obtained within a specific volumetric space in the concrete.

We believe the post-drilling process involves many dependencies, special considerations, and extreme care. With little tolerance for error, this process may lead to misleading results putting the installer at higher risk of installation failure, expensive repairs, and making project decisions based on potentially flawed data. We have invented a cast-in-place device that meets the volumetric space requirements of ASTM F2170 and does not encounter similar issues as noted with the current post-installed sensor. A cast-in-place device eliminates the issues related to drilling and cleaning the concrete hole with the post-installed sensor.

### PRIOR ART

In the construction industry today, the pace of construction is ever-increasing. Concrete flooring is common in many commercial and residential buildings. Various types of floor coverings are used, including carpeting, wooden flooring, vinyl tiles, epoxies, polyurethanes, and polymer-terrazzo. Flooring such as these is typically sensitive to concrete with high retained moisture. Flooring installed over concrete with high moisture tends to fail over time, leading to fungi and mold growth underneath the floor coverings.

ASTM F1869 “Standard Test Method for Measuring Moisture Vapor Emission Rate of Concrete Subfloor Using Anhydrous Calcium Chloride” was the test method used to measure the vapor transmission on the surface of the slab. This required a calcium chloride kit to be placed on the floor's surface and a protective dome to be placed over the kit and sealed. The kit is left under the dome for a specified time, then removed and weighed. The emission rate is then calculated based on the difference between the original weight of the kit and its final weight.

ASTM F2170 “Standard Test Method for Determining Relative Humidity in Concrete Floor Slabs Using in situ Probes” is the test method used to determine the relative humidity in concrete with an electronic humidity sensor. The ASTM specifies a volumetric space needed to determine the relative humidity.

Typically, the sensor is installed between 16 to 36 hours after concrete placement. The installation requires a hole to be drilled with a specific size diameter drill bit. The depth of the hole is project-specific. The hole is then cleaned with a vacuum hose to remove the debris or with air jets to blow out the drill debris. The post-installed sensor is inserted into the drill hole. The sensor is manufactured with deformable circumferential fins to ensure the measuring space is sealed. Alternately, the sensor can be fitted with an extension tube to prevent any premature relative humidity loss from the exposed side wall of the drill hole. The top of the hole or the pipe is then capped.

Nilsson (2018), reports that these probes cannot be installed after the temperature effect as a result of the drilling of the hole is small. This takes several days. Taking readings in a hole too early after drilling can result in an error of greater than 10%.

The drilling process not only generates heat but can also cause microcracking of the concrete if not done with care. Additionally, the drilling process combines concrete paste and fractured aggregate within the required volumetric measurement space. Concrete is comprised of 70 percent aggregate, and depending on the type of aggregate used in the concrete mix, the measurements obtained will depend on the aggregate present in the hole.

A summary of disadvantages of the prior art are:

The interval between drilling the hole and installing the sensor is essential to obtain good data. Installing the sensor too early can lead to errors in the readings obtained.

Destructive drilling generates heat and accelerates evaporation of the drilled hole surface.

Destructive drilling will result in the exposed face of the hole consisting of concrete paste and fractured aggregate. The dissipation of relative humidity from the concrete paste is different from that of the dissipation of relative humidity from the fractured aggregate. Since concrete is made up of 70% aggregate, the type and volume of the aggregate present in the volumetric space will differ. This makes obtaining accurate relative humidity readings very dependent on the aggregate of the mix.

Cleaning the hole with jets of air to force the dust out of the hole will change the environment in the hole. Any change will require the concrete around the hole to equalize to the surrounding concrete.

The level of cleaning of the drilled hole. An improperly cleaned hole may increase the rate of reduction in the measured relative humidity readings.

The walls around the drilled hole may be micro-cracked because of the drilling process. The presence of microcracks will result in a rapid change in relative humidity.

The fit created by the circumferential fins around the sensor housing into the drilled hole is dependent on the uniformity of the drilled hole and how carefully the sensor was installed. This may result in an increased rate of reduction in the measured relative humidity readings.

When a reading is taken, the protective cap must be removed at the top of the hole. Removing the protective cap will change the relative humidity in the hole with the reader probe inserted.

Physically contacting the sensor to obtain the reading requires the removal of the sealing cap and insertion of a reader probe. This may result in an increased rate of reduction in the measured relative humidity readings.

The seal on the extension tube can affect the relative humidity reading collected. This may result in an increased rate of reduction in the measured relative humidity readings.

Research has shown that the environment in the drilled hole takes approximately 48 to 72 hours to equilibrate and stabilize.

### BACKGROUND OF THE INVENTION

Our invention relates to the invention of a cast-in-place relative humidity concrete sensor. The sensor has a housing, which protects the delicate circuitry of the embedded sensors placed in fresh concrete and still allows one to measure the relative humidity of concrete, ensuring the humidity levels in concrete are appropriate for installing floor coverings.

Concrete is comprised of approximately 70% aggregate. The essential components of concrete comprise cement, water, and fine and coarse aggregate. When the components are mixed, the concrete mix must be workable enough to place concrete into the forms and finish the concrete surface. The paste structure at this point is saturated because of the mix-water required to make the concrete workable and allow uniform placement into the concrete forms. Therefore, when measuring the relative humidity of the concrete at this early stage, most of the relative humidity will be dissipated from the concrete paste.

### SUMMARY OF THE INVENTION

Our invention improves the process of measuring the relative humidity in newly placed concrete from the time the concrete is placed into the form. The relative humidity measurements are not affected by drilling a hole to install the sensor, the presence of fractured aggregate in the volumetric space, environmental stabilization due to drilling, cleaning process, etc.

The housing allows gasses and sound to pass through readily while protecting the device from debris and liquid ingress due to the fine grain structure and surface tension on and within the grain structure (Boyle's Law). This creates a protective air volume within the walls of the housing. At the center of the housing protective air space is/are the sensing device(s). The housing can be any geometric shape with a single opening into which device(s) may be sealed; however, spherical shapes are more suited for submersion applications due to gravity's physics, which reduces air pockets.

When measuring the relative humidity in newly placed concrete, the objective is to determine the relative humidity in the concrete paste structure and not a combination of concrete paste and fractured aggregate that may be present on the walls in the volumetric space measurement obtained. Any destructive means required to install a device after the concrete has cured, such as hammer drilling, results in a disruption of the concrete structure, microcracking, exposing, or fracturing the aggregate and paste structure. It is also not required to clean the drilled hole from dust, either by shooting a jet of air into the hole or vacuuming the hole, thus changing the environmental condition in the hole. The DEVICE, since it is positioned in the concrete prior to the time of concrete placement, does not disrupt the concrete structure. The DEVICE will be surrounded by concrete paste and not a combination of concrete paste and fractured aggregate. The relative humidity measurements will not be a result of the concrete paste and not be influenced by any fractured aggregate.

Comparing the relative humidity measurements obtained from the cast-in-place device to one that requires drilling, cleaning, sealing, and then installing the post-installed sensor, the relative humidity recorded shows that the dissipation rate differs. The difference implies that there are external factors that influence the measurements from the post-installed sensor. The external factors can include improper sealing, the volume of fractured aggregate present around the sensor, debris from the drilling process may be present in the hole, or the loss of relative humidity due to improper capping at the top of the hole, etc.

Our invention is installed in the formwork before the concrete is placed, typically on a reinforcing bar, before the concrete is placed. Our invention will be able to communicate and transfer any collected measurements wirelessly to the mobile app. Our invention does not require any drilling, cleaning, or other destructive means for installation.

Objects And Advantages

Accordingly, our patent offers several advantages over the prior art. Several objects and advantages of our invention are:

No destructive drilling is required.

No external heat is generated during drilling to accelerate the evaporation of the drilled hole surface.

Measurements can be taken immediately without having to wait for the environment to equalize.

Measurements are obtained from the concrete paste around the sensor.

Measurements are not influenced by fractured aggregate.

Cleaning of the drilled hole is not required.

There is no microcracking influencing the readings obtained.

The sensor is secured prior to the placement of concrete.

The sensor is encased completely during the concrete placement.

Readings are obtained wirelessly.

The readings can be obtained immediately after the concrete is placed and monitored long-term.

Further, our invention's advantages, usefulness, and functionality will become apparent by referencing the figures and detailed descriptions listed below.

### BRIEF DESCRIPTION OF DRAWINGS

The figures show a cross-section of the sensor and the housing. It shows the protective housing that protects the sensor while simultaneously allowing the sensor to measure the environment.

### DETAILED DESCRIPTION OF THE INVENTION

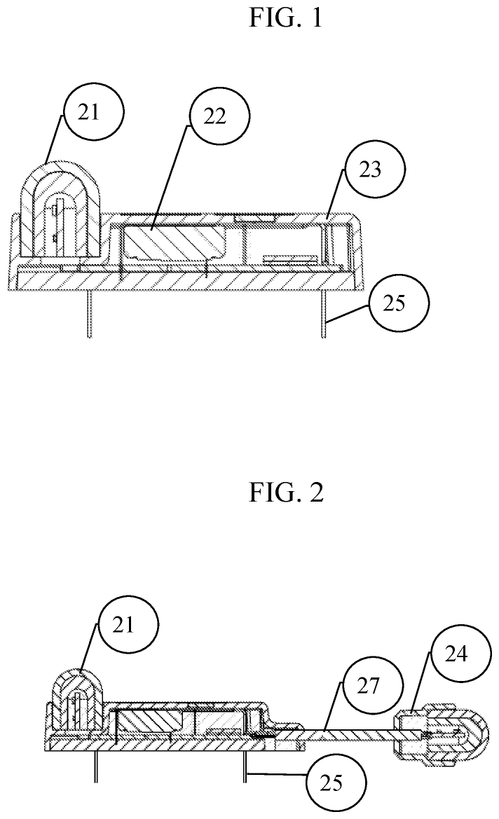

FIG. 1 is a cross-sectional view of our invention with different components in the sensor.

FIG. 2 is a cross-sectional view of our invention with a remote probe attached vis a wire lead with different components in the sensor and remote probe.

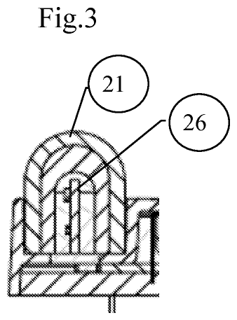

FIG. 3 is a cross-section view of housing around the sensor circuit board in my invention

### REFERENCE NUMERALS IN DRAWINGS

21 protective housing
22 power supply
23 embedded measuring device
24 remote measuring unit
25 tie wire
26 sensing board
27 wire lead

We claim:

1. A device for measuring the environment in the concrete comprising:
(a) a measuring unit that protects electronic components from environmental degradation.
(b) said protecting housing unit that extends past the unit body.
(c) said remote probe, which will be an extension of said unit body.

2. The device, according to claim 1, wherein said protective housing consists of at least one layer of porous containment formed in any geometric shape.

3. The device, according to claim 1, wherein said device is embedded into the environment in which measurements are recorded.

4. The device, according to claim 1, wherein said housing meets the required volume in ASTM F2170.

5. The device, according to claim 1, wherein said housing:
(a) protects said sensor chip and printed circuit board.
(b) allows said device to be cast into concrete.

6. The device, according to claim 1, wherein said device can:
(a) be installed without the need to create access to the point where measurements are taken.
(b) start collecting data on the environment without any change in the environment caused by drilling or installation of any external tubing.
(c) start collecting data from the time the concrete is placed.

7. The device, according to claim 1, wherein
(a) said measurements obtained are from the concrete paste structure and not influenced by a combination of concrete paste and fractured aggregate. (b) no external protective covering is needed to stabilize the measured environment.

8. The device, according to claim 1, wherein said measurements obtained is not influenced by:
(a) any external means to clear or clean the pathway to the sensor.
(b) any microcracking within the measuring environment.
(c) by debris in a drilled hole.
